(19) United States
(12) Patent Application Publication
(10) Pub. No.: US 2013/0171079 A1
(43) Pub. Date: Jul. 4, 2013

(54) COMPOSITION TO STABILISE KOJIC ACID

(71) Applicants: Elliot Isaacs, Elstree (GB); Daniel Isaacs, Elstree (GB)

(72) Inventors: Elliot Isaacs, Elstree (GB); Daniel Isaacs, Elstree (GB)

(73) Assignee: Pangaea Laboratories Ltd., Elstree (GB)

(21) Appl. No.: 13/728,973

(22) Filed: Dec. 27, 2012

(30) Foreign Application Priority Data
Dec. 28, 2011 (GB) GB1122393.0

(51) Int. Cl. A61K 31/351; A45D 40/26; A61K 38/54; A61K 38/44; A61Q 17/04; A61Q 19/02

(52) U.S. Cl. CPC A61K 31/351; A61Q 17/04; A61Q 19/02; A61K 38/54; A61K 38/446; A61K 38/443; A45D 40/261
USPC 424/59; 424/62; 424/94.2; 424/94.4; 514/460; 132/317

(57) ABSTRACT

A novel composition is disclosed to provide stable kojic acid in a cosmetically or pharmaceutically acceptable medium using a synergistic combination of a sulphur dioxide releasing agent such as sodium metabisulphite and one or more catalases, catalase mimetics, superoxide dismutases, and superoxide dismutase mimetics.

### CROSS-REFERENCE TO RELATED APPLICATION

The present non-provisional patent application claims the benefit of priority of foreign Patent Application No. GB1122393.0, which is entitled A COMPOSITION TO STABILISE KOJIC ACID, and which was filed Dec. 28, 2011 which is incorporated in full by reference herein.

### FIELD OF THE INVENTION

The present invention relates to a composition to stabilize kojic acid.

### BACKGROUND OF THE INVENTION

There are several routes to lightening skin using cosmetic or pharmaceutical compositions. kojic acid is a popular ingredient of these compositions as it blocks several parts of the melanogenetic pathway simultaneously, creating significant results in a variety of skin types.

The principle problem with using kojic acid is stability. The molecule is highly reactive and degrades within weeks in solution. The process is accelerated by heat, UV, or low pH. Degraded kojic acid has little or no function for skin lightening and is a distinctive yellow colour.

Prior solutions to the stability issue have included airless packaging to remove most of the oxygen, coloured packaging to prevent UV exposure, and unfortunately, cream formats to disguise the yellow discolouration. These options are far from ideal.

One useful, if obvious, solution to the problem would be to add a preservative such as sodium metabisulphite. Sodium metabisulphite is in a class of preservatives that release sulphur dioxide. This increases the stability of kojic acid noticeably, keeping discolouration to a low level for up to two months. However this option is not enough for two reasons. Firstly, two months is not sufficient shelf life for a cosmetic or pharmaceutical composition. Secondly, a preferable format for a skin lightening composition would also include one or more other lightening ingredients, such as lactic acid, niacinamide and ascorbic acid. The net effect of these ingredients may lower the pH, and accelerate the degradation of kojic acid. This has been shown in experiments to overcome the stabilising effect of sodium metabisulphite during a two month period.

Several alternative stabilisers were suggested and tested without success. It was then hypothesised that reducing agents or antioxidants may help by preventing free radical formation. Various antioxidants were tested without noticeable effect. A new hypothesis suggested that catalytic or enzymatic antioxidants such as catalases, superoxide dismutases, and/or mimetics of catalases or superoxide dismutases may overcome the problem as they would function continuously for long periods of time. Surprising experimental results showed they worsened the problem—a solution of kojic acid and propyl gallate, a superoxide dismutase mimetic, degraded significantly more than a solution of kojic acid alone after two weeks.

Even more surprisingly, the combination of sodium metabisulphite and propyl gallate stabilised a kojic acid solution for more than two months. Researchers expected the combination to deliver better results that propyl gallate, but worse results than sodium metabisulphite alone. Instead the kojic acid solution remained completely clear for more than two months. The result is certainly not additive and therefore must be due to some previously unknown synergy between sodium metabisulphite and propyl gallate.

### BRIEF SUMMARY OF THE INVENTION

In one embodiment, the invention may be a composition comprising of kojic acid, a sulphur dioxide releasing compound and one or more antioxidants in a cosmetically or pharmaceutically acceptable medium. The antioxidants may be catalytic or enzymatic antioxidants such as catalases, catalase mimetics, superoxide dismutases, and superoxide dismutase mimetics.

In another embodiment the combination of a sulphur dioxide releasing compound such as sodium metabisulphite and one or more catalases, catalase mimetics, superoxide dismutases, and superoxide dismutase mimetics act synergistically to prevent the kojic acid from degrading.

Various concentrations of kojic acid are considered in the range of 0.00001% to 40%, but particularly 0.5%, 1%, 2% and 4%.

All compounds that release sulphur dioxide are considered, however a preferred embodiment may comprise sodium metabisulphite in a concentration in the range between 0.00001% and 30% of the composition. Optimal embodiments may consider concentrations of 0.001%, 0.05%, 0.2%, 1% and 3%.

Any superoxide dismutase or superoxide dismutase mimetic may be considered, but preferred embodiments comprise propyl gallate, gallic acid and derivatives of gallic acid, dimethyl methoxy chromanol, and/or superoxide dismutase in a concentration in the range between 0.00001% and 30%. Also considered are any catalases or catalase mimetics, but a preferred embodiment may comprise copper PCA (copper salt of pyrrolidone carboxylic acid) in a concentration in the range between 0.00001% and 30%. Optimal embodiments may consider concentrations of these catalytic or enzymatic antioxidants of 0.0001%, 0.01%, 0.1% and 2%.

As the composition may be used for skin lightening, any other skin lightening component may be considered in addition to kojic acid. An exemplary composition would include one or more of lactic acid, niacinamide, thiotic acid, hydroquinone, ascorbic acid or a vitamin c derivative, L-leucine, and a form of arbutin.

The composition may further comprise any other pharmaceutical or cosmetically active agent (defined as a natural or synthetic compound that has a cosmetic or therapeutic effect on the skin, hair or nails including but not limited to lightening agents, darkening agents, anti-acne agents, shine control agents, anti-microbial agents, anti-inflammatory agents, anti-mycotic agents, anti-parasite agents, external analgesic or anaesthetic agents, sunscreens, photo-protectors, antioxidants, keratolytic agents, detergents or surfactants, moisturisers or humectants, nutrients, vitamins, energy-enhancers, growth factors, anti-perspiration agents, astringents, deodorants, hair-removers, firming agents, anti-callous agents and agents for hair, nail and/or skin conditioning.) Of particular interest are curcumin, taurine, plant sterols, pine bark extract, green tea, red tea, white tea, horsetail extract, marine cartilage, caffeine, kieslerde, copper peptides, copper pyrrolidone carboxylic acid (copper PCA) euk-134, copper(II) 3,5-diisopropylsalicylate, minoxidil and other natural or synthetic nitric oxide donators, finasteride, dutasteride, spironolactone, superoxide dismutase (and mimetics), dimethyl methoxy chromanol (Lipochroman-6), catalase mimetics, saw palmetto and other natural and synthetic anti-dihydrotestosterone agents, hydrolysed lupine protein, vitamins c, a, e, b, f, h, k (and derivatives), bacterial filtrates, glucosamine sulphate, or any combination of these.

As a cosmetic or pharmaceutical composition the invention may be embodied as an anhydrous powder, an aqueous solution, an oil-based solution, a suspension, an oil-in water mixture with or without a surfactant, a gel, or a cream. In any of the above embodiments the composition may comprise two or more parts, to be used in series or mixed together prior to use. Each part may have the same physical embodiment, or take different physical forms. Use of each part in series may be separated by a period of time from seconds up to 6 weeks as part of a course of applications.

It is considered that any of the above embodiments may be used in combination with other procedures. In one aspect the composition may be used in conjunction with one or more devices and/or other composition(s) to improve transdermal penetration of one or both compositions. Exemplary devices for this purpose comprise an array of microneedles such as a microneedle roller, an electrophoresis apparatus, an ultrasound emitter, an unfractionated laser, a fractionated laser, and an iontophoresis apparatus. Exemplary compositions considered may comprise surfactants; keratolytic agents such as salicylic acid; physical and chemical exfoliants; superficial, medium and deep chemical peels.

A further considered embodiment may combine the composition with one or more additional skin lightening method(s), device(s) and/or composition(s). Any skin lightening method, device, or composition is considered, but preferred embodiments comprise compositions to reflect or absorb UV, commonly known as sunscreens; lactic acid; niacinamide; thiotic acid; hydroquinone; ascorbic acid or a vitamin c derivative; L-leucine; a form of arbutin; keratolytic agents such as salicylic acid; physical and chemical exfoliants; superficial, medium and deep chemical peels. A preferable device in this embodiment comprises one or more microneedle arrays which may be in the form of a roller.

Yet another embodiment considered comprises of providing multiple containers of the composition, or parts of the composition in a kit. The kit may comprise one or more containers of the composition with one or more additional cosmetically or pharmaceutically active compositions, which may also lighten the skin. Additionally or alternatively, the kit may comprise one or more compositions for use with certain devices to be used in combination with the composition, with or without the device itself, and/or replacement components for the device. One exemplary kit may comprise of the composition with one or more of a sanitising or sterilising solution, an analgesic or anaesthetic composition, a microneedle roller, a replacement microneedle array for the microneedle roller, a moisturising composition, and a composition a sunscreen.

### BRIEF DESCRIPTION OF THE DRAWINGS

No drawings are submitted with the application as none are necessary with this disclosure.

### DETAILED DESCRIPTION OF THE INVENTION

The present invention is described with respect to particular but the invention is not limited thereto but only by the claims.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may refer to different embodiments. Furthermore, the particular features, structures or characteristics of any embodiment or aspect of the invention may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practised without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may, in some embodiments, mean only one. The term “about” can mean “approximately”.

A test was conducted to measure the effect of sodium metabisulphite and propyl gallate on the stability of kojic acid in aqueous cosmetic or pharmaceutical compositions.

In this regard kojic acid is known to change colour from a slight yellow to very dark yellow as it degrades, and so colour change was used as the indicator of stability vs. degradation.

All samples contained 2% kojic acid in an aqueous cosmetic base. Different combinations of sodium metabisulphite and propyl gallate were used in each sample as shown in Table 1.

TABLE 1
Combinations of sodium metabisulphite and propyl gallate added
to 2% kojic acid and an aqueous solution to form 4 test samples.

| | Sodium | |
|---|---|---|
| Sample | Metabisulphite | Propyl Gallate |
| A | | |
| B | 0.2% | |
| C | | 0.1% |
| D | 0.2% | 0.1% |

Each sample was photographed and optically assessed against a 20-stage yellow colour spectrum, where 1 was a very pale straw yellow, and 20 was a dark yellow. Photographs were taken on day 1, day 7 and day 42. In between photographs, the samples were stored together at normal room temperature.

As can be seen, after 42 days at room temperature kojic acid was almost completely degraded in some samples.

TABLE 2
Results of degradation of kojic acid from day 1 to day 42,
where “1” is no degradation and “20” is completely degraded.

| Sample | Day 1 | Day 42 |
|---|---|---|
| A | 1 | 19 |
| B | 1 | 17 |
| C | 1 | 20 |
| D | 1 | 2 |

The addition of sodium metabisulphite alone improved stability slightly over 42 days. The addition of propyl gallate alone worsened stability slightly over 42 days.

Prior to the results it would have been predicted that the combination of propylgallate and sodium metabisulphate would give an additive score of 18 or 19. However the combination of the two significantly stabilises kojic acid, producing a much lower degradation score. This indicates significant synergy between the two molecules.

What is claimed is:

1. A composition comprising kojic acid, sodium metabisulphite and one or more catalases, catalase mimetics, superoxide dismutases, and superoxide dismutase mimetics in a cosmetically or pharmaceutically acceptable medium.

2. A composition according to claim 1, wherein the sodium metabisulphite is present in a concentration in the range between 0.00001% and 30%.

3. A composition according to claim 2, wherein the sodium metabisulphite is present at a concentration of about 0.2%.

4. A composition according to claim 1, wherein the superoxide dismutase mimetic is selected from the list of propyl gallate, gallic acid and derivatives of gallic acid.

5. A composition according to claim 4, wherein the propyl gallate, gallic acid and/or derivative of gallic acid is present in a concentration in the range between 0.00001% and 30%.

6. A composition according to claim 5, wherein the propyl gallate, gallic acid and/or derivative of gallic acid is present at a concentration of about 0.1%.

7. A composition according to claim 1, wherein the superoxide dismutase mimetic is dimethyl methoxy chromanol.

8. A composition according to claim 7, wherein the dimethyl methoxy chromanol is present in a concentration in the range between 0.00001% and 30%

9. A composition according to claim 1, further comprising one or more additional components to lighten the skin.

10. A composition according to claim 9, wherein the additional component(s) to lighten the skin are one or more of lactic acid, niacinamide, thiotic acid, hydroquinone, ascorbic acid, L-leucine, and a form of arbutin.

11. A composition according to claim 1, wherein the composition comprises two or more parts.

12. A composition according to claim 11, wherein the two or more parts are mixed prior to use.

13. A composition according to claim 11, wherein at least one of the parts is an anhydrous powder.

14. The composition according to claim 1, provided as part of a kit comprising one or more of a sanitising or sterilising solution, an anaesthetic composition, a microneedle roller, a replacement microneedle array for the microneedle roller, a moisturising composition, and a sunscreen composition.

15. Use of a composition according claim 1, wherein the sodium metabisulphite and one or more of a catalase, a catalase mimetic, a superoxide dismutase, and a superoxide dismutase mimetic are used to stabilise kojic acid in solution.

16. Use of a composition according to claim 1, in conjunction with a microneedle roller.

17. Use of a composition according to claim 1, in conjunction with one or more additional skin lightening method(s), device(s), and/or composition(s).
